(19) United States
(12) Patent Application Publication
(10) Pub. No.: US 2006/0189917 A1
(43) Pub. Date: Aug. 24, 2006

(54) Implant for draining chamber water from the front eye chamber into the episcleral veins

(75) Inventors: Winfried Mayr, Modling (AT); Clemens Vass, Vienna (AT); Ewald Unger, Vienna (AT)

Correspondence Address: PROSKAUER ROSE LLP, 1001 PENNSYLVANIA AVE, N.W.,, SUITE 400 SOUTH, WASHINGTON, DC, 20004, US

(73) Assignee: AUSTRIA WIRTSSERVICE GELLSCHAFT MIT, Vienna (AT)

(21) Appl. No.: 10/542,404

(22) PCT Filed: Dec. 23, 2003
(86) PCT No.: PCT/AT03/00381

§ 371 Date: Mar. 20, 2006

(30) Foreign Application Priority Data
Jan. 23, 2003 (AT) A 88/2003

(51) Int. Cl. A61F 9/007

(52) U.S. Cl. 604/9

(57) ABSTRACT

A drainage implant for draining chamber water from the eye, comprising a tube-shaped part (1) which has at least one lumen, whose open distal end (3) can be introduced into an episcleral vein and whose open proximal end (2) can be introduced into the front eye chamber for draining the chamber. The proximal and center areas of the tube are covered with silicon. Said covering is embodied in the center area in the form of a plate (6) with eyelets (6) in order to stabilize and fix the implant by means of sutures on the sclera. The implant contains a guide wire (8) inside the tube (1), the front of said guide wire being acutely sharpened. The guide wire is used to puncture the vein and also stabilizes the very thin tube (1) for implantation.

The present invention relates to an implant for draining chamber water from the front eye chamber into the episcleral veins, for use in glaucoma, consisting of the measures listed in the generic part of claim 1.

Glaucoma is a disease which is characterized by chronically progressing lesions in the optic nerve with the chief risk factor of elevated intraocular pressure. Approximately 2:1 of chamber water are produced per minute in the interior of the eye and drain through the trabecular mechanism situated in the front ocular chamber into the canal of Schlemm, and from there through the chamber water veins into the venous system. The main physiological resistance to this chamber water drainage lies in the juxtacanicular portion of the trabecular meshwork, i.e. between the front eye chamber and the canal of Schlemm. In the case of chronic open angle glaucoma, even this resistance is pathologically elevated.

Fistulating glaucoma operations (trabeculectomy) presently represent the standard procedure in operations for lowering ocular pressure in glaucoma patients. The chamber water in that case is drained through a wound in the sclera from the front eye socket under the connective tissue and carded away. While the short-term successes of about 90% are acceptable, in the course of a few years the healing of the wound leads not rarely to closure of the fistula and thus to late failure of the operation.

Another drainage mode for chamber water uses cyclodialysis. In this operation about 3 mm is removed from the limbus of the sclera, the sclera is severed and a spatula is introduced through it between the sclera and the ciliary body beneath it. This spatula is pushed forward up to the corner of the chamber and thus the front chamber of the eye is opened. Thus a connection between the front chamber and the gap between the ciliary body and sclera (i.e., the supracillary gap). Although the success rates of this operation are acceptable, the technique has been neglected on account of the very frequent and poorly controllable hypotonia ft entails.

New operative techniques of non-penetrative glaucoma surgery (deep sclerectomy, viscocanalostomy) have lately demonstrated that the canal of Schlemm can be repeatably represented in chronic open-angle glaucoma, and furthermore it can also be used functionally at least in viscocanalostomy. In EP 0 898 947 A2 an implant was reported which is implanted in the canal of Schlemm in connection with a viscocanalostomy for permanent extension into the canal of Schlemm. In the deep sclerectomy, a fistulation under the connective tissue is sought, because in some cases the attempt is made to support this by the use of implants. Nevertheless, these methods were unable to solve the problem of postoperative scarring, so that the medium-term success rates are similar to those of trabeculectomy. It is common to all methods for the non-penetrative glaucoma surgery that a thin layer of tissue, also called a trabeculo-descemetic window, remains intact and exercises a not precisely definable and also variable effect on the resistance to drainage (Dietlein T S, Graefe's Arch Ophth 2000).

An improvement of the chamber water drainage from the front chamber into the canal of Schlemm, and simultaneously keeping the canal of Schlemm open, is the purpose also of the apparatus described in WO 00/13627 A1. Here a stent is implanted in the canal of Schlemm, which stretches the trabecular mechanism and has openings directed as the trabecular mechanism.

In complicated cases, e.g., after multiple preliminary operations, drainage implants have long been used (Molteno, Krupin, Schocket, Baerveldt, Ahmed) which are all constructed on the same principle: a thin tube (usually of silicone) is introduced with its open end into the front eye chamber, drains the water to a plate or cerclage band affixed to the back of the pupil. Around this plate or the cerclage band a capsule eventually forms, while the resistance to flow (and thus the intraocular pressure) is determined by the permeability of this capsule as well as the surface of the capsule. These methods also involve the problem of scarring.

Spiegel describes (1999) in connection with cadaver eyes, a method for draining from the front eye chamber directly into the canal of Schlemm. He used for this purpose a silicon tube with an outside diameter of 0.15 mm and an inside diameter of 0.05 mm.

In International Patent Application WO 00/64393 A1 an implant is described for draining the chamber water from the front eye chamber into the canal of Schlemm. It can be introduced with its open proximal portion into the front eye chamber, on the one hand, and on the other hand with the distal portion into the canal of Schlemm on both sides.

Both in Spiegel's work and in the international patent cited above, the problem of the stable fixation of the drainage implant remains unsolved. A solution for this has been given in Patent WO 02/087479 A2.

For the majority of eyes suffering from glaucoma, the direct implantation of a drainage implant into the canal of Schlemm appears to be a usable method. While this might offer the advantage of a physiological drainage way, such a solution cannot be considered in some patients. Above all in the case of pre-operated eyes, but also in many forms of glaucoma, as for example in the case of pseudo exfoliation glaucoma, the canal of Schlemm might be partially obliterated, or at least impaired in its operation (Ritch R. Surv Opthalmol 2001; 45:165). In these cases a direct drainage into the eplscleral veins might be helpful.

It is the object of the present invention to create an implant for the drainage of chamber water from the front eye chamber into the episcleral veins.

To solve this problem the implant of the invention has the features set forth in the specific part of claim 1.

The invention is described hereinafter by exemplification without limiting the general idea of the invention, by means of embodiments with reference to the drawings, wherein:

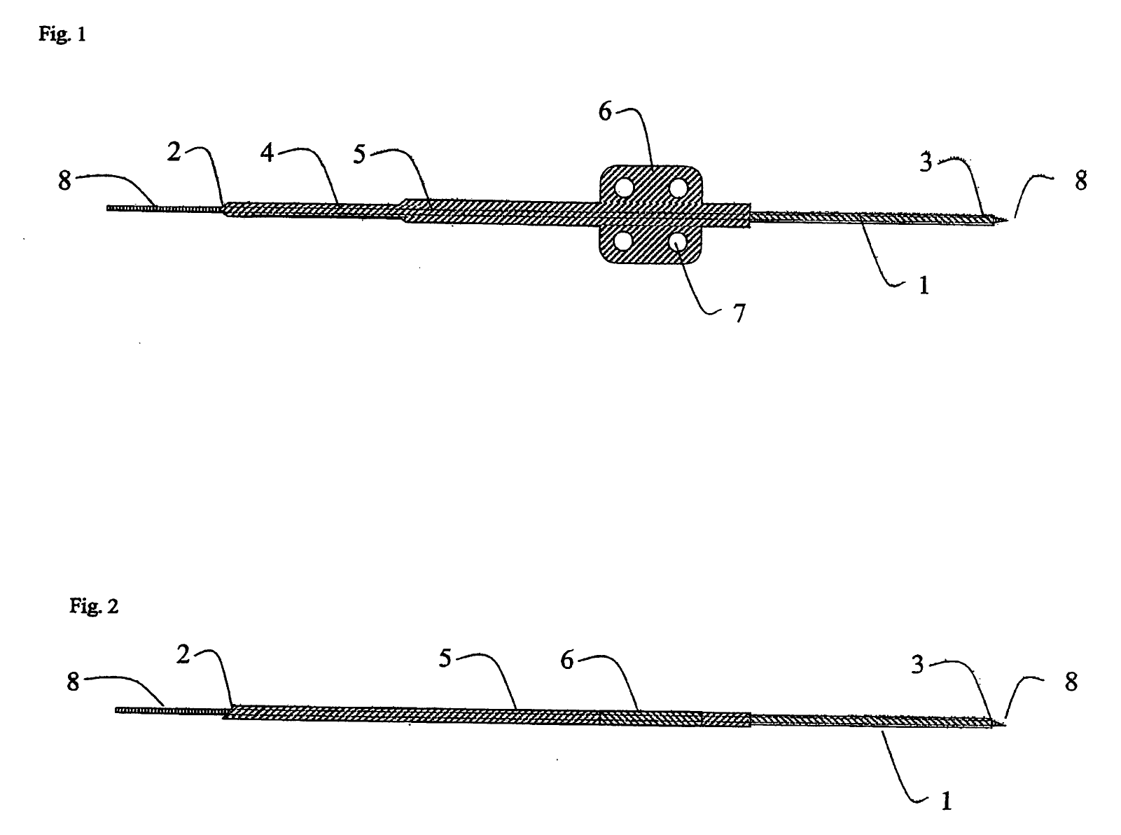

FIG. 1 shows a schematic representation of a top plan view of a drainage implant corresponding in part to the present invention;

FIG. 2 shows a schematic representation of a side view of a drainage implant corresponding in part to the present invention;

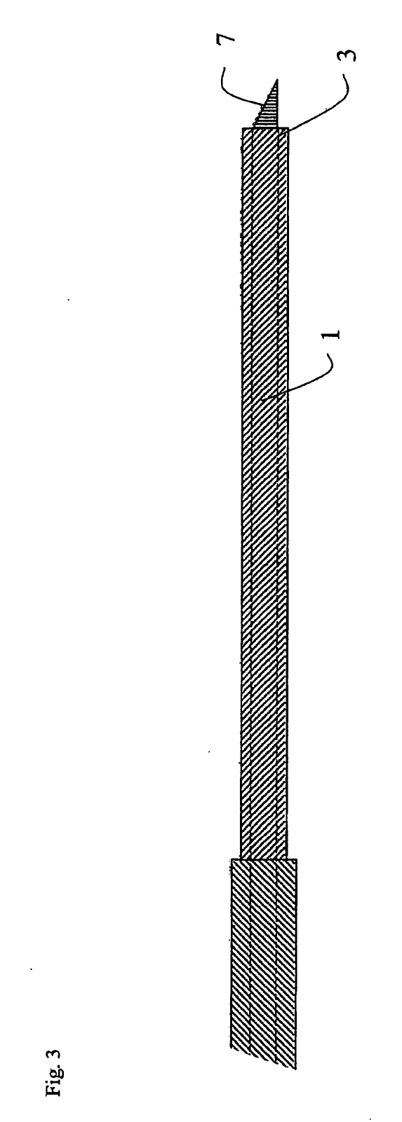

FIG. 3 shows a schematic representation showing a detail view of the distal end of a drainage implant corresponding partially to the present invention;

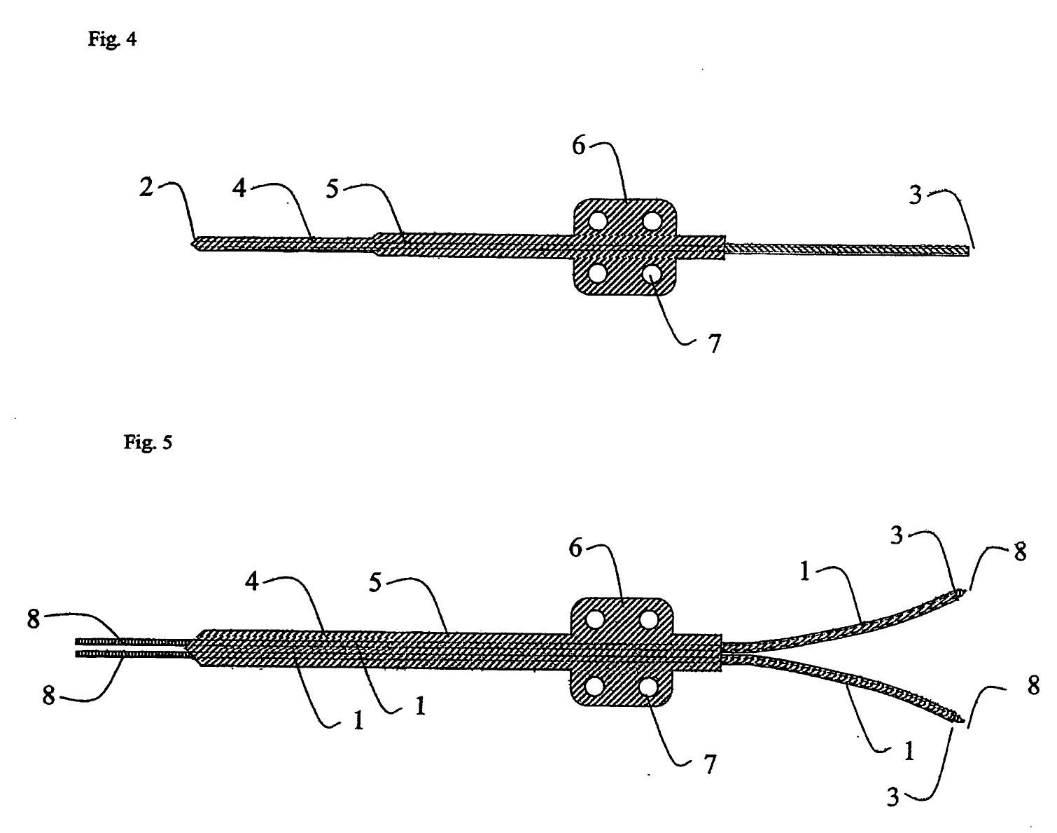

FIG. 4 shows a schematic representation showing a drainage implant corresponding partially to the present invention after removal of the guide wire; and

FIG. 5 shows a schematic representation showing a drainage implant corresponding partially to the present invention, in an embodiment with two tubular parts.

In what follows, a preferred variant of the invention is explained in detail. The present invention is directed to an implant for the drainage of chamber water from the front eye chamber into the episcleral veins.

FIGS. 1-4 show an embodiment of the present invention of the implant with a tubular portion 1 which has at least one lumen and can be introduced with its open proximal end 2 for drainage of the chamber water into the front eye chamber, and can be introduced with its open distal end 3 into an episcleral vein. In the proximal area 4 and in the center area 5 of the implant, the tube 1 is jacketed in plastic, this jacketing being formed in the center area 5 as a plate 6 with eyelets 7 to permit the stabilization and fixation of the implant by means of sutures.

For the implantation of the tube 1 into an episcleral vein, the implant contains inside of the tube 1 a guide wire 8 with a sharp front end, which on the one hand stabilizes the very thin tube 1 and with which secondly the vein is pierced.

In operation, the conjunctiva is opened, an episcleral vein is uncovered and pierced with the tip of the guide wire 8. After the punctio the tube 1 is pushed forward with its distal end 3 into the vein and then affixed with the eyelets to the sclera with sutures. Then the guide wire 8 is removed and the proximal end 2 of the implant is introduced through a limbic puncture incision of suitable diameter into the front chamber of the eye. The proximal area 4 and the central area 5 of the implant can be shifted under a scleral flap.

FIG. 5 shows an embodiment in which the proximal area 4 and the central area 5 of the implant are in the form of double-lumen tubes. These branch at the distal end 3 into two separate tubes 1 which can be introduced each into an episcleral vein. In the proximal area 4 and in the central area 5 the double tube is encased in plastic, and in the middle area this casing is in the form of a plate 6 with the eyelets 7 in order to permit the implant to be stabilized and affixed with stitches.

1. Implant for draining chamber water from the front eye chamber into one or more episcleral veins, consisting of at least one tubular part (1), characterized in that, to drain the chamber water into an episcleral vein, this tubular part (1) is provided with a guide wire (8) stabilizing it and provided with a distal sharp tip in order that, after the piercing of the particular vein and withdrawal of the guide wire, it can be brought with its distal outlet end (3) into the episcleral vein, while the proximal inlet end (2) can be introduced into the front eye chamber.

2. Implant according to claim 1, characterized in that the proximal (4) and the central area (5) contain several tubular parts (1) which branch out in the distal area and can be implanted with their distal ends (3) into a plurality of episcleral veins, these tubular parts (1) containing each a guide wire (8) for piercing the particular episcleral veins (FIG. 5).

3. Implant according to claim 1, characterized in that the open distal end or ends (3) of the tubular part (1) are rounded or beveled so as to be able to introduce them atraumatically into one or more episcleral veins.

4. Implant according to claim 1, characterized in that the diameters of the lumina forming the tubular parts (1) are between 0.02 mm and 0.1 mm.

5. Implant according to claim 1, characterized in that the tubular portion (1) is encased at least partially with silicone or other appropriate plastic.

6. Implant according to claim 1, characterized in that at the tubular portion (1) a plate (6) is applied which can contain one or more eyelets (7) for fixation by stitching.

7. Implant according to claim 1, characterized in that at least parts of the implant are made from biocompatible material, for example silicone or other appropriate plastics, from stainless steel, from titanium, from a noble metal such as silver, gold or platinum, or from a biological material.

8. Implant according to claim 1, characterized in that at least parts of the implant are coated with suitable material to produce a desired biological reaction or to prevent an undesired biological reaction.

9. Implant according to claim 1, characterized in that a check valve is provided in the tubular portion (1), by which retrograde flow of blood into the front eye chamber is prevented.
